(19) United States
(12) Patent Application Publication
(10) Pub. No.: US 2007/0044805 A1
(43) Pub. Date: Mar. 1, 2007

(54) Method for controlling a ventilator and ventilation device

(76) Inventors: Wolfgang Wedler, Hamburg (DE); Mark Brandmeier, Hamburg (DE); Karl Andreas Feldhahn, Hamburg (DE)

Correspondence Address: Friedrich Kueffner, Suite 910, 317 Madison Avenue, New York, NY, 10017, US

(21) Appl. No.: 11/509,834

(22) Filed: Aug. 24, 2006

(30) Foreign Application Priority Data
Aug. 26, 2005 (DE) 10 2005 040 654.8
Mar. 17, 2006 (DE) 10 2006 012 320.4

(51) Int. Cl. A61M 16/00; A62B 9/06; A61B 5/08

(52) U.S. Cl. 128/207.14; 600/529

(57) ABSTRACT

A method for controlling a ventilator, wherein at least one ventilation-dependent parameter is measured and evaluated by a control unit for the control of a ventilator. At least one operating parameter of the ventilator is changed as a function of the measured parameter. At least one constituent of the blood of the user is measured noninvasively as the ventilation-dependent parameter.

### BACKGROUND OF THE INVENTION

1. Field of the Invention

The present invention relates to a method for controlling a ventilator, wherein at least one breathing-dependent parameter is measured and evaluated by a control unit and at least one operating parameter of the ventilator is varied as a function of the measured parameter.

The invention also pertains to a ventilation device, which has a control unit, a breathing gas source, and at least one sensor for detecting a breathing-dependent parameter.

2. Description of the Related Art

Methods and devices of this type are used, for example, to optimize the operating behavior of ventilators. The goal of such optimization can be, for example, to minimize power consumption, to minimize the noise produced, to maximize user comfort, or to maintain defined ventilation parameters within narrow tolerances. Applications in the medical area can include, for example, bilevel, PSV/PCV, and VCV ventilation and other forms and types of ventilation, including CPAP and APAP ventilation. Other applications in the area of medical devices can also be found in mobile or stationary units for supplying patients with oxygen or with oxygen-enriched air.

Various applications outside the area of medical devices are also possible. In the case of breathing equipment for divers or firemen, for example, it can be important to minimize the power consumption when the unit is powered by batteries or to minimize background noise so as not to impair acoustic perception. The same problem can also occur in the industrial area in the case of activities which require the use of breathing equipment for protection of the person in question from the effects of gases in the environment and during which the persons in question do not have their hands free, because of the nature of their activities, to operate their breathing equipment themselves. Finally, applications are also conceivable in the area of ventilators for use by astronauts or pilots.

In the case of patients with respiratory failure, the key parameter for stabilization is the carbon dioxide partial pressure PaCO₂. The goal of ventilation is to ventilate the patient into the eucapnic range, that is, for example, to use the ventilator to ensure the patient's ability to exhale CO₂. “Eucapnia” is usually understood to mean a PaCO₂ value of 40 mm Hg (pCO₂<45 mm Hg).

According to the state of the art, an invasive blood gas analysis is conducted to determine the PaCO₂ value required to stabilize the patient (titration) during ventilation. For this purpose, a blood sample is taken before and after the ventilation, and the blood values SaO₂, PaO₂, PaCO₂, and pH as well as the concentration of the base excess (BE), temperature, metabolites, and electrolytes are determined.

This method suffers from several disadvantages:
the procedure is invasive and therefore causes the patient pain;
blood gases are determined only at certain times;
the procedure is labor-intensive and time-consuming;
the procedure is limited to clinical use; and
there is a considerable cost factor for the analysis (centrifuge, equipment, etc.).

### SUMMARY OF THE INVENTION

The object of the present invention is to improve a method of the type indicated above in such a way that at least one target parameter of the ventilation process can be maintained within narrow tolerances.

This task is accomplished according to the invention in that, as the breathing-dependent parameter, at least one parameter of the patient's blood is determined noninvasively.

As a variant, this parameter is determined by means of a sensor positioned on the user's skin.

As another variant, this parameter is determined by means of a sensor for analyzing the expiratory air.

As parameters of the blood, constituents can also be considered blood parameters.

In particular, it is also possible for at least one constituent of the blood of the user to be measured noninvasively by means of a sensor positioned on the patient's skin and to use this value as the breathing-dependent parameter.

Another task of the invention is to design a device of the type indicated above in such a way that it can be operated easily with only a few manipulations while offering wide-ranging functionality at the same time.

This task is accomplished according to the invention in that the sensor is designed to be placed on the user's skin and to measure noninvasively at least one parameter of the user's blood.

Through noninvasive determination, the blood further under ventilation, it is possible to obtain trend values quickly for the analysis of the effectiveness with which the ventilator has achieved the desired stabilization and to avoid the disadvantages of a conventional blood gas analysis.

Through noninvasive determination of the blood values, especially the CO₂ value, and their transmission to the control unit of the connected ventilator, the ventilation parameters are adjusted automatically by the ventilator in such a way that a predetermined target carbon dioxide partial pressure is reached.

The ventilator is given complete or limited autonomy to reach the predefined blood gas target values within a predefinable bandwidth of the minimum and maximum limits of the ventilation settings (safety function). The ventilation setting under which the target value has been reached is retained by the unit as long as the target CO₂ value remains within the tolerance range.

To prevent an implausible automatic ventilation setting (e.g., hyperventilation, hypoventilation, barotrauma/volutrauma, etc.), maximum corridors for the parameters are predefined (f, Ti:T, IPAP, EPAP, Vt, etc.). These can also be monitored by way of alarms.

When the target CO₂ value is no longer being reached, the ventilator automatically adjusts the settings as necessary within the permitted bandwidths. This can be relevant during changes of position, different stages of sleep, changes in the impedance of the lungs, etc.

Through the use of the inventive method and device, it is possible to ventilate patients into the eucapnic range, which is a PaCO₂ of 40 mm Hg (pCO₂<45 mm Hg). This value is the goal for every patient, but it cannot be reached in every patient, because many patients become habituated to their hypercapnia, which may have persisted for a long time. For this reason, the target CO₂ value can also be set and reset by the physician.

One embodiment of the inventive device therefore has a ventilation auto-titration function with specification of the CO₂ target parameter. For example, patients with hypercapnia (>50 mm Hg at rest) and mild COPD are ventilated under the following parameters:
volume setting: 900±100 mL
respiratory rate: 20±3 breaths/min
positive pressure respiration: 25±9 mbars

This is used preferably for the stabilization of a patient who requires long-term ventilation so that, by partial or complete transfer of responsibility for the patient to the ventilator, the physician can be freed and costs can thus be lowered. The device can be started with the settings preselected by the physician, and after the values have been recorded for a certain period of time and the ventilator has automatically made the necessary changes, the settings in question can then be given to the patient, who can then use them at home.

To improve the response to slow changes in the status of the patient's condition, i.e., changes which require a resetting of the device, a measuring means for determining the pCO₂ value is placed on the patient not only while he is under the supervision of the physician but also during ventilation at home. This can be placed on the ear, for example, on the finger, or any other suitable place on the body. Communication between the measurement means and the device can proceed on the basis of various principles: cable connection, radio link, infrared, Bluetooth®, mechanical, electrical, etc.

The device records the changes, and if the changes in the patient's pulmonary status or in the patient's condition are critical, it will generate a warning message with instructions to contact the attending physician. Alternatively, in the case of certain patients, the physician can also allow an automatic change in the parameters of the ventilator. In an expanded form of the invention, the message to the physician can also proceed directly via an interface (telephone system, mobile telephone, or other data-transmission alternative).

For certain types of diseases, the preselected settings present at system start-up can be replaced by, for example, permanently predefined settings stored in the ventilator. Thus the physician can select the disease condition directly on the display, and the device will take into account the important settings and their bandwidths characteristic of this disease and implement the settings for the disease in question.

In addition to the pCO₂ measurement, a noninvasive measurement of the oxygen content can also be performed, leading perhaps to the administration of oxygen to improve the patient's condition. This can be done continuously, as an on-demand system, by means of bolus administrations or by some other type and form of administration.

The series of experiments which were conducted to test the invention showed the surprising result that a significant reduction in the pCO₂ value to the eucapnic range (pCO₂<45 mm Hg) was achieved in 18 of 20 patients.

Because transcutaneous CO₂ measurement is now technically mature and commercially available, and because the measured values are close to the PaCO₂ values, it is proposed according to the invention that the technology available from, for example, SENTEC (SenTec AG, Ringstrasse 39, 4106 Therwil, Switzerland) (V-Sign System) be used to implement the method in the ventilator. This technology is described in, for example, EP 1 335 666 and in EP 1 535 055.

For adult patients, the SENTEC sensor system makes it possible to place the measuring probe on the ear for an extended period of time and thus provide satisfactory accuracy of the PtcCO₂ trend values and low usage cost at the same time. The technology offers significant advantages over the previously established measuring technology available from Radiometer based on diffusion electrodes. For example, there is no wear or oxidation of the electrodes, the drift is small, and the system can be calibrated easily/automatically. In one embodiment of the invention, an OEM solution of SENTEC sensors can be connected to the ventilator.

A measurement technology should be adopted in the form of sensors, ear clips, and a membrane oscillating function for calibration. All monitoring functions, settings of the capnographic parameters/alarm limits, and the software itself are implemented in the ventilator. The power supply and control of the CO₂ monitoring system are integrated into the firmware of the ventilator or interlinked with it.

The invention can also be implemented in modular form. The basic unit, i.e., a ventilator according to the state of the art, is connected mechanically and electrically by means of an optional component. The ventilator notices in such cases that a module is connected and automatically switches into the required mode.

In addition to SENTEC, the company Kontron (Kontron AG, Oskar-von-Miller-Strasse 1, 85386 Eching/Munich, DE) also offers comparable OEM solutions, which can be integrated into ventilators. The Tosca Company offers already established sensors, which can be used for this purpose.

In addition, Linde Medical Sensors AG offers a sensor, which functions as follows: Oxygen and carbon dioxide can diffuse through the human skin. A sensor heated to about 43-44° C. is used. As a result of the increase in blood circulation, especially in the upper layers of the skin, and the sweating of the skin as a natural reaction to the heat, it is easier for gases—in this case O₂ and CO₂—to diffuse between the skin and the surface of the skin, and the gases thus arrive at the surface of the sensor. It should be made explicit that other technologies for noninvasive measurement of the PaCO₂ value can also be interlinked/coupled to ventilators to make it possible to determine the PaCO₂ value, on the basis of which the ventilator is then controlled to reach a target PaCO₂.

According to the invention, it is also possible to determine the CO₂ content of the exhaled air and to use that, as an indirect determination of the PaCO₂, for the control function. The spectroscopic measurement of end-tidal CO₂, for example, is suitable for this purpose.

In addition, the use of both technologies, i.e., the determination of the CO₂ content of the exhaled air and the transcutaneous determination of the pCO₂ content, can be used in conjunction with the device, so that, for example, pathological changes in lung tissue (e.g., onset of pulmonary edema, mucous catarrh, etc.) can be established on the basis of the differences between the two measurements. For example, a high pCO₂ and a low SpCO₂ can point to a pumonary gas exchange disturbance.

According to the invention, it is also possible to determine noninvasively other blood values such as SaCO, SaO₂, pH, bicarbonate (HCO₃) concentration, base excess (BE), and hemoglobin concentration and to use these values to control the ventilator.

The invention describes a method for controlling a ventilation device, where at least one blood value or at least one value associated with blood values is determined, where at least one target value can be specified for this value, and where the device is then controlled in such a way that the measured value is brought closer to the target value.

The invention also describes a method for controlling a ventilation device, where a measurement value which is indicative of the carbon dioxide concentration in the blood is determined, where at least one target value can be specified for the measurement value, and where the device is then controlled in such a way that the determined measurement value is brought closer to the target value.

The concentration of the constituent can be measured, for example, by measuring a fractional content of the parameter in the blood.

So that a parameter which is especially sensitive to ventilation can be detected, it is proposed that the carbon dioxide content of the blood be measured.

The specified values can be accurately maintained by the use of a feedback control circuit, which adjusts the operating parameter of the ventilator. The blood parameter data are transmitted as setpoints and actual values to this circuit.

An even better automatic control concept can be achieved by adopting a strategy, by adapting it as a function of the measurement results and the type of ventilation, and by using this strategy for automatic control. According to an especially advantageous embodiment, strategies can be named and/or assigned to certain diseases. Thus the device makes available stored strategies together with their settings and bandwidths for special disease conditions and can respond optimally to them. These special strategies are permanently defined in the device. In addition, there are also free strategies, which the physician can configure, name, and store according to his own ideas.

The adaptability can be made even better in that a mode selection is performed when the operating parameter is changed.

Operator-independent operation is supported by allowing the control unit to change the operating parameter autonomously.

To take into account external control specifications, it is proposed that operator inputs be evaluated by the control unit.

The accuracy with which setpoints can be maintained can be improved by designing the control unit to evaluate measurement data from the sensor continuously.

The amount of analysis required can be reduced by designing the control unit to analyze the measurement data from the sensor only during predefined time intervals.

Mounting the sensor on a bandage creates a wide range of choices for the selection of a suitable location for the sensor.

The speed with which the sensor can be positioned can be improved by mounting the sensor on a clip.

The ease with which the device can be operated can be improved by locating the clip on the user's finger.

It is has been found advisable, especially for mobile applications, for the clip to be mounted on the user's ear.

The sensor can be placed on any part of the body with good circulation. Good locations include, for example, the earlobes, the tips of the fingers, the temples, and the forehead as well as the area of the nose. To achieve a significant increase in the ease of use and wearing comfort, a transcutaneous sensor can, in a preferred embodiment, be embedded in the forehead support of a mask being used for ventilation and sleep therapy.

Other preferred positions are the temples. To position the sensor, either the forehead support or the straps for holding the mask in place can be used. For this purpose, the sensor can be positioned along two axes (x, y). The sensor can also be integrated into the edge of the mask.

Because the sensor is attached to the mask or in and on its straps, this arrangement makes it possible to install the electrical connection between the sensor and the device at a point near the ventilation hose. The cable used to connect the sensor could optionally be embedded in the hose, attached externally to the hose, or integrated externally. If the measuring and control channel connections with the mask are integrated into the ventilation hose, only a single plug-and-socket connection is required. If a cordless transmission unit is integrated into the sensor, the acquired data can be compressed and then transmitted to the evaluation unit in the device and thus enter into the control and regulation process.

According to another proposal, medications can also be administered via the ventilation hose on the basis of the recording of the PaCO₂ signal according to the same pattern. Thus the special physiological condition of the patient can be effectively addressed. The medications can be administered in various ways, including by the use of a humidifier or mister.

The various features of novelty which characterize the invention are pointed out with particularity in the claims annexed to and forming a part of the disclosure. For a better understanding of the invention, its operating advantages, specific objects attained by its use, reference should be had to the drawing and descriptive matter in which there are illustrated and described preferred embodiments of the invention.

### BRIEF DESCRIPTION OF THE DRAWING

In the drawing:

FIG. 1 is a perspective view of a ventilation device consisting of the basic unit, the breathing gas hose, and the breathing mask;

FIG. 2 is a schematic functional block diagram which illustrates the implementation of a control method;

FIG. 3 is a detailed functional block diagram of the implementation of control of the device with automatic or selectable adaptation of functional modes and automatic control strategies;

FIG. 4 is a detailed functional block diagram in further documentation of the “strategy use” functional block units in FIG. 3;

FIG. 5 is a functional block diagram corresponding to FIG. 4 for application of an automatic strategy;

FIG. 6 shows a sensor mounted on the ear of a user;

FIG. 7 shows a sensor mounted on the finger of a user;

FIG. 8 shows a display;

FIG. 9 shows a user wearing a breathing mask; and

FIG. 10 shows a breathing mask with identification of areas suitable for the attachment of a sensor.

### DETAILED DESCRIPTION OF THE INVENTION

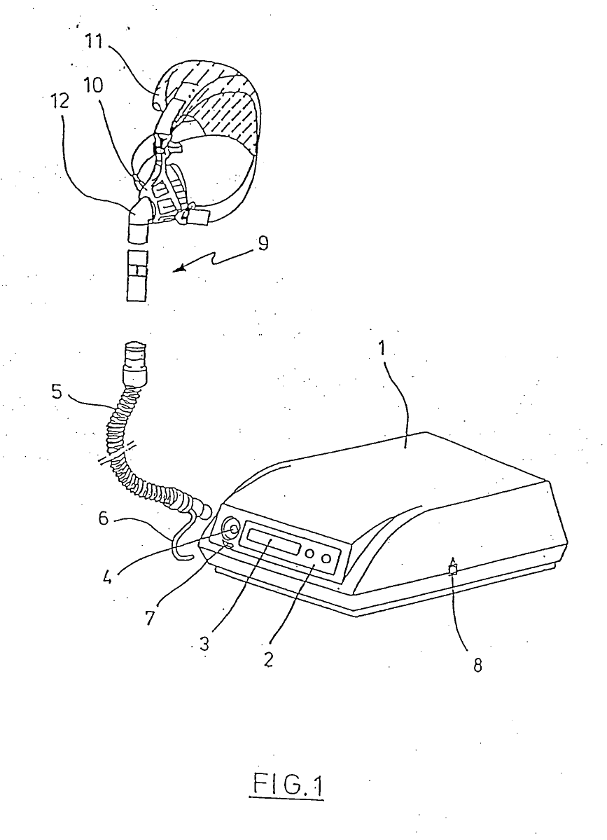

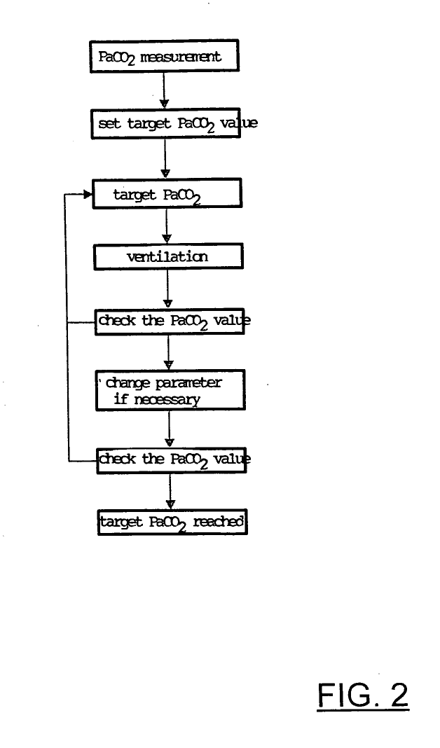

FIG. 1 shows the basic design of a ventilation device. In the unit housing (1) with the control panel (2) and display (3), a breathing gas pump is installed in the interior of the unit. A connecting hose (5) is connected to the ventilator by means of a connecting element (4). An additional pressure-measuring hose (6), which can be connected to the unit housing (1) by means of a pressure inlet connector (7), can extend along the connecting hose (5). The unit housing (1) has an interface (8) to allow the transmission of data. A humidifier can also be adapted to fit the device.

At the end the connecting hose (5) facing away from the unit housing (1), an exhalation element (9) is provided. An exhalation valve can also be used.

FIG. 1 also shows a patient interface in the form of a breathing mask (10), which is realized as a nasal mask. The mask is held in position on the patient's head by means of a hood (11). On the side facing the connecting hose (5), the patient interface (10) has a connecting element (12).

The blood value sensor can be connected to the ventilation device via the interface (8). The interfaces can be connected by cables or designed in the form of an infrared interface, a Bluetooth interface, or a USB interface. In particular, the connection between the blood value sensor and the ventilator can be realized electrically, pneumatically, optically, mechanically, or by combinations of these variants. In the area of the unit housing, an oxygen feed valve can be adapted to fit the ventilator. The breathing gas can also be enriched with oxygen to improve the care of the patient.

In addition, interfaces to third-party devices and data management systems for copying to storage media, for connecting to an ECG, EEG, printer, defibrillator, etc, can also be provided in one of the devices.

By means of a modem or other interface, furthermore, recorded data such as trends, unusual events, warnings, etc., can be transmitted to the physician, and conspicuous occurrences, hours of operation, or other data ensuring satisfactory function can be sent to the maintenance/customer service department as needed.

According to the invention, it is also possible to apply the methods and means described in the field of emergency medicine. Although capnometry is already being used here according to the state of the art, there is still no communication between the capnometry and the emergency ventilator. So that this can be guaranteed in the future, especially when patients are being transported, it is desirable in this sector as well to ventilate the patient to a target value. Here, however, the mature technology of pulsoxymetry, also in combination with capnography, can be used advantageously. By taking advantage of the “Air-Mix” setting on an emergency/transport ventilator, the ratio of oxygen to ambient air can be adjusted to save oxygen, only a limited amount of which can usually be carried along in a compressed gas cylinder.

To achieve the conservation effect, it would be possible to control and to administer the oxygen supply as a function of the oxygen saturation either throughout or only during the initial inhalation phase. The O₂ can therefore be administered on this basis and on the basis of the monitoring of the SaO₂ and the resulting calculation of the difference between SaO₂ and the target SaO₂.

According to another embodiment, a full-face mask or an endotracheal tube can be used. The interface (13) is provided for a connection to the sensor, which is provided to measure PCO₂, SpO₂, the pulse, or other blood gas values.

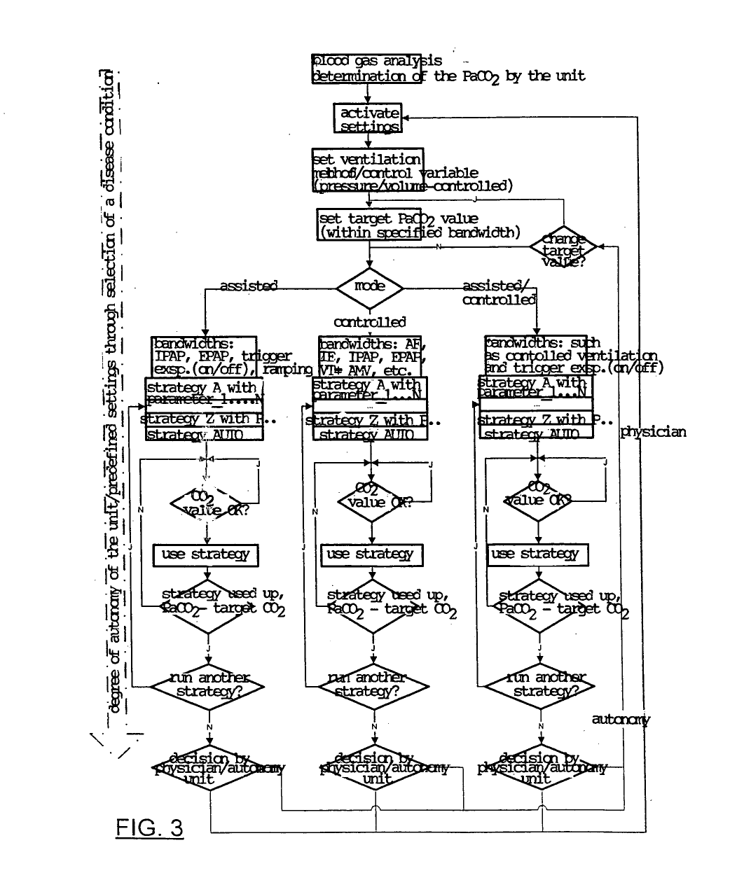

The implementation of the method is explained by way of example on the basis of FIG. 3. During the titration of a patient on a ventilator with ventilation to a target value, the patient's blood is either subjected to a blood gas analysis or the PaCO₂ value is determined by means of the previously explained and listed possibilities and methods.

A disease condition can now be directly selected externally, and the device can be granted the desired degree of autonomy over the following decisions. The data which are required for the unit's autonomy and which are used as, for example, settings, bandwidths, minima, and maxima, are stored in the unit and are read out as needed. Thereupon, the unit asks for a decision concerning the ventilation method (pressure-controlled or volume-controlled ventilation). The unit can then ask for the target CO₂ value.

In the next step, the unit asks whether the patient should be ventilated in an assisted manner, in a controlled manner, or in an assisted/controlled manner. Now, depending on the degree of autonomy, it can be decided externally or by the unit which parameters are to be set, what their bandwidths are to be, and what the maxima and minima should be for the ventilation parameters or whether it is best to use one of the strategies on file. Each of these strategies contains a priority list of 1 to N different settings and is processed within the scope of their bandwidths.

The PaCO₂ at a specific moment can be requested, for example, and this can be compared with the target CO₂ value, so that a decision concerning the further processing of the prioritized bandwidth can be made. If the bandwidth has been completely used up, the parameter possibly following next in the strategy is adjusted until the target CO₂ value for the patient has reached the optimum setting. If the strategy has been completely processed but the target CO₂ value has still not been reached, either a new strategy can be selected, a new target CO₂ value can be set, a new ventilation method/control variable (pressure-controlled/volume-controlled) can be selected, or a new mode (assisted and/or controlled ventilation) can be set to achieve a further improvement in the patient's condition. This decision can be communicated to the user either by an alarm and/or requested or executed independently by the unit.

Alternatively, the target value being aimed at can also be bracketed within a bandwidth, so that, although the unit has a goal which it can reach, it can consider the current settings permissible if the strategies have been exhausted. The target value and the target value bandwidth can be make part of the unit settings.

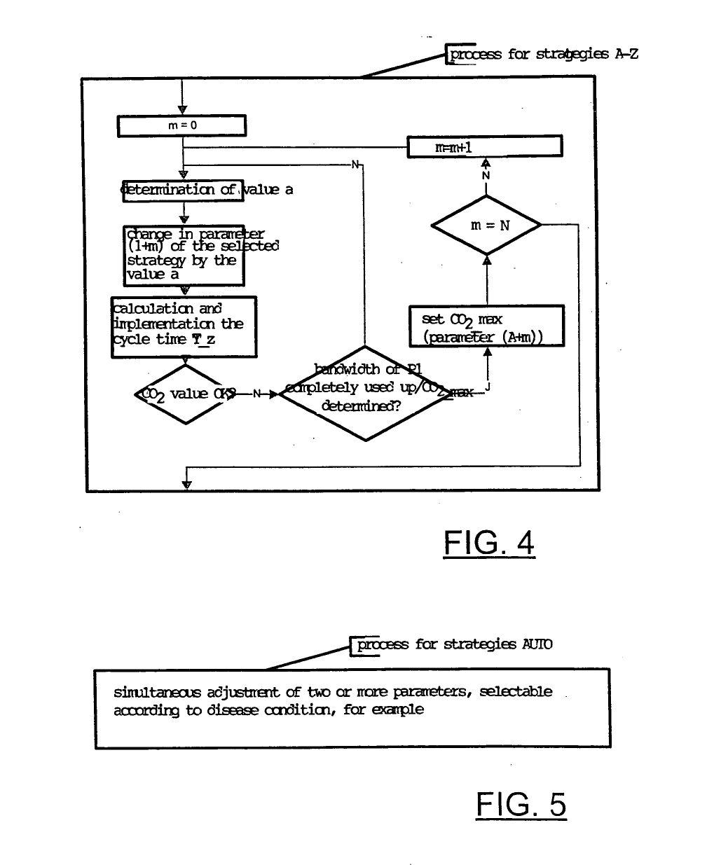

To achieve a high degree of accuracy with which the settings can be reached and to increase the speed at which they can be reached, the intensity “a” of the change and the cycle time “T_z” are calculated or requested in a manner specific to the program or read out from internal memory. The change intensity “a” determines the variable “change of the current parameter”, whereas the cycle time determines the length of time between changes in the parameter in question, as shown in FIGS. 4 and 5. This takes into account the fact that the CO₂ value requires a certain amount of time to settle, and each patient reacts differently to changes in the ventilation parameters. The two values are therefore calculated from the data specific to the patient and to the unit. The change intensity can, for example, depend on the cycle time and on the difference between the PaCO₂ value and the target CO₂ value and on other characteristic values formed from the time periods relevant to ventilation.

It is also possible to graduate the change intensity and to subject it to threshold formation. The change intensity is adjusted depending on the threshold which has been crossed in the downward direction.

So that titration can be carried out efficiently, it is also possible to rely on the principle of the self-learning machine (artificial intelligence, neuronal network). The data are acquired and analyzed continuously and are used to improve the adjustment of the parameters, thus leading to a more rapid titration of the patient with a specific disease condition. This learning and adjustment process can be done independently in each unit, or the data can be collected centrally with the help of the interfaces and copied over to the other units.

It is generally known that the CO₂ value can be improved primarily by way of the pressure, so that this is also the means of choice for the above proposal. The other settings will be oriented around this one and/or relegated to secondary status.

With the help of this intelligent form of ventilation, it is possible for the first time to establish a link between blood values and the associated ventilation setting. This means a decisive improvement with respect to time, cost, and quality in the ventilation setting and its monitoring.

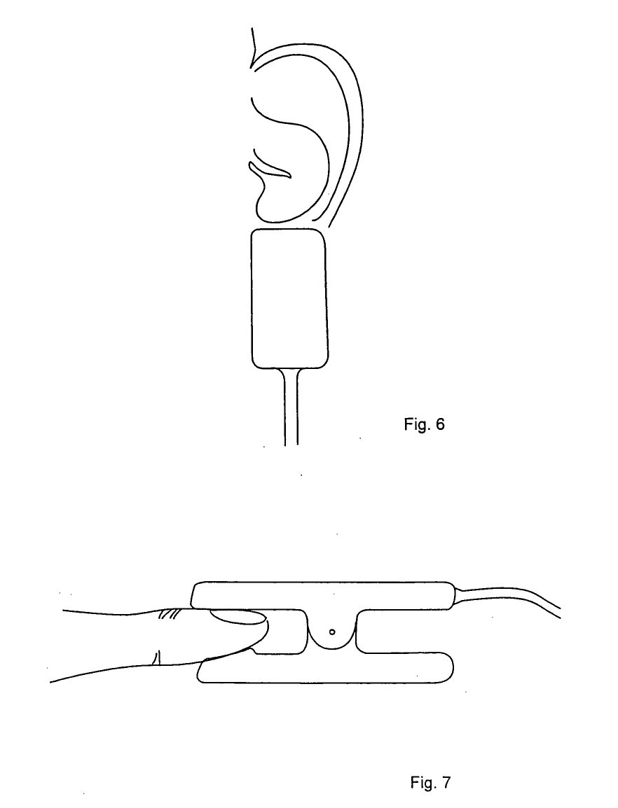

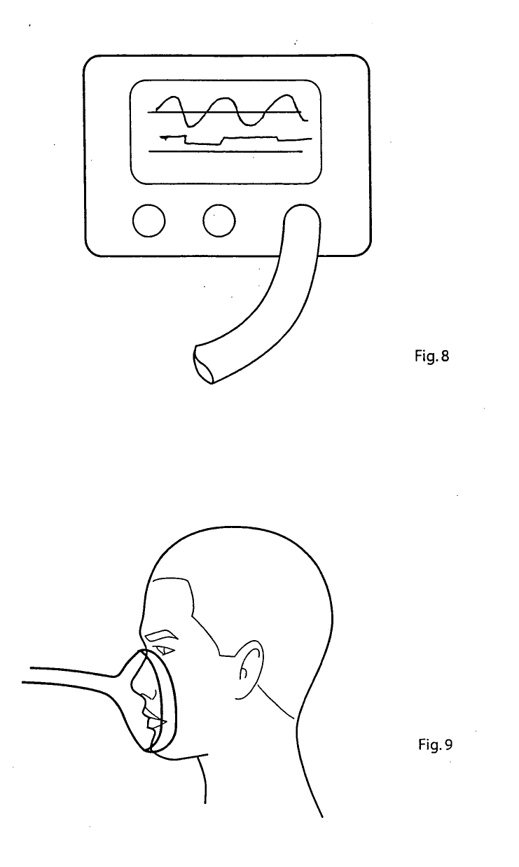

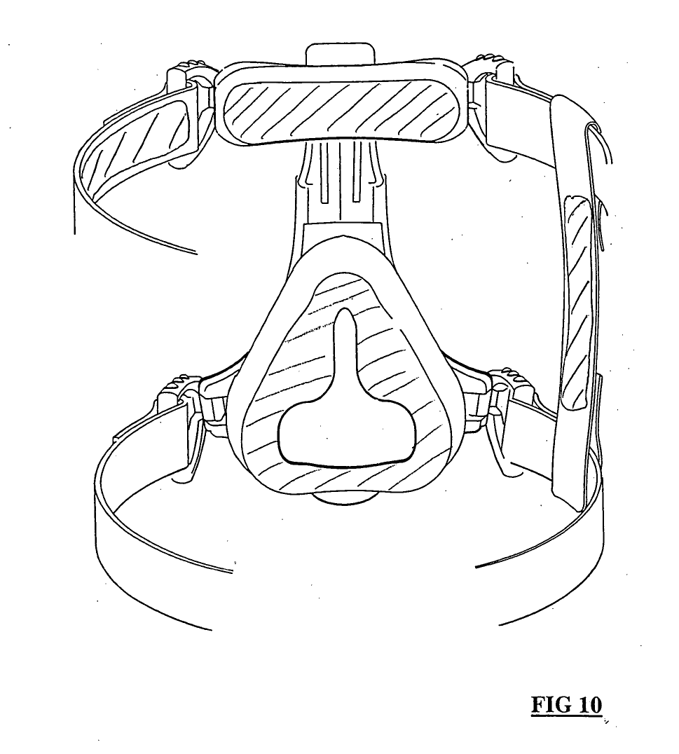

Additional exemplary embodiments can be seen in FIGS. 6 and 7. The sensor can be attached either to the ear or to the fingertip of the patient. In addition, the ventilator according to FIG. 8 can be equipped with a monitor for showing the current settings, either as values or as a curve, and the patient data as determined by the unit. FIG. 9 shows the use of the breathing mask by a patient.

Another exemplary embodiment which allows the sensor to be placed close to the skin can be seen in FIG. 10. Here all the areas on which a sensor can be placed, for example, are shaded.

While specific embodiments of the invention have been shown and described in detail to illustrate the inventive principles, it will be understood that the invention may be embodied otherwise without departing from such principles.

1. A method for controlling a ventilator, the method comprising measuring at least one breathing-dependent parameter and evaluated in the parameter by a control unit, and changed at least one operating parameter of the ventilator as a function of the measured parameter, further comprising determining at least one parameter of the blood of the user noninvasively as a breathing-dependent parameter.

2. A method according to claim 1, wherein the breathing-dependent parameter is measured noninvasively by at least one sensor positioned on the skin of the patient.

3. A method according to claim 1, comprising determining a fractional content of the parameter in the blood.

4. A method according to claim 3, wherein the measured fractional content of the blood is measured indirectly by analysis of the expiratory gas.

5. A method according to claim 1, wherein the content of carbon dioxide in the blood is determined.

6. A method according to claim 1, wherein the content of oxygen in the blood is determined.

7. A method according to claim 1, wherein the content of hemoglobin in the blood is determined.

8. A method according to claim 1, wherein the content of components of the hemoglobin in the blood is determined.

9. A method according to claim 1, further comprising changing the operating parameter of the ventilator within an automatic control circuit, wherein the data on the constituents of the blood are supplied as setpoints and actual values.

10. A method according to claim 1, wherein, to implement the automatic control, strategies are adjusted as a function of the measurement results.

11. A method according to claim 1, wherein a mode selection is carried out when the operating parameter is changed.

12. A method according to claim 1, wherein the operating parameter is changed autonomously by the control unit.

13. A method according to claim 1, wherein operator inputs are evaluated by the control unit.

14. A method according to claim 1, wherein measurement data of the sensor are evaluated continuously by the control unit.

15. A method according to claim 1, wherein the control unit evaluates measurement data of the sensor only within predefined time intervals.

16. A method according to claim 1, wherein a strategy is assigned to a certain disease condition.

17. A method according to claim 1, wherein stored strategies and corresponding settings and bandwidths are assigned to specific disease conditions.

18. Ventilation device comprising a control unit, a breathing gas source, and at least one sensor for detecting a breathing-dependent parameter, wherein the sensor is configured for the noninvasive measurement of at least one constituent of the blood of the user.

19. A device according to claim 18, wherein the sensor is configured to be placed on the skin of the user.

20. A device according to claim 18, wherein the sensor is configured to perform an indirect measurement of the constituent of the blood by analysis of the expiratory gas.

21. A device according to claim 18, wherein the sensor is positioned on a bandage.

22. A device according to claim 18, wherein the sensor is positioned on a clip.

23. A device according to claim 22, wherein the clip is adapted to be positioned on a finger of the user.

24. A device according to claim 22, wherein the clip is adapted to be positioned on the ear of the user.

25. A device according to claim 18, wherein the sensor is positioned on the edge of the breathing mask.

26. A device according to claim 18, wherein the sensor is positioned on straps of the breathing mask.
